(19) United States
(12) Patent Application Publication
(10) Pub. No.: US 2009/0149767 A1
(43) Pub. Date: Jun. 11, 2009

(54) SYSTEM FOR DETECTION AND PROCESSING OF PHYSICAL PARAMETERS OF HUMAN BODY AND METHOD THEREOF

(76) Inventor: Walter Rossetti, Milano (IT)

Correspondence Address: DAVID A. GUERRA;INTERNATIONAL PATENT GROUP, LLC, 2025 17TH AVENUE N.W., CALGARY, AB, T2M 0S7, CA

(21) Appl. No.: 11/719,870

(22) PCT Filed: Nov. 28, 2005
(86) PCT No.: PCT/IT2005/000696

§ 371 Date: May 22, 2007

(30) Foreign Application Priority Data
Nov. 30, 2004 (IT) MI2004A 002305

(51) Int. Cl. A61B 5/0408; H04M 1/00

(52) U.S. Cl. 600/522; 455/556.1

(57) ABSTRACT

What is disclosed is a system and a method for the detection and processing of physical parameters of the human body, which comprises a plurality of sensors which can be applied to the skin of a subject. Said system comprises at least an interface circuit for the processing of the signals coming from said sensors and at least a reception/transmission circuit for the transmission of the signals processed by the interface circuit.

### TECHNICAL FIELD

The present invention refers to a system and a method for the detection and processing of physical parameters of the human body. The invention refers in particular, but not exclusively, to the detection and processing of signals of an electrocardiogram of a patient. Systems for telemedicine have been known for a long time and continuously evolve; said systems for telemedicine allow patients who suffer from particular pathologies to be remotely monitored by means of telecommunication equipment. Said telecommunication equipment allows transmitting the signal detected by particular sensors applied to a patient to a diagnostic centre of choice.

Nevertheless, several pathologies can arise without any notice also in subjects considered basically healthy. In particular, several cardiac problems may suddenly arise and, if not promptly faced, may constitute a serious danger for health, or even for life itself. Such an event may happen not only to people subjected to particular strain conditions, like athletes or other sports people during sports competition or training, but also to people in everyday life.

Time is therefore a relevant factor in order to be able to help and try effective treatments to a subject struck, for example, by a heart attack. In fact, it is deemed that a prompt intervention, which is tried within 20 minutes since arising of a heart attack, could allow limiting damages due to such a pathology or even saving a person's life. Nevertheless, a person who is considered healthy, and for this reason is not normally subjected to a remote monitoring of his health conditions, is often unable to report the arising of such problems. For this reason, the need to provide to everyone a practical and easy-to-use system, which allows a quick reporting of significant information on the nature of the problems to a remote unit exists. In general, the object of the present invention is to provide a system for detection and processing of physical parameters of the human body that can be available to a person in order to report possible health problems to a remote unit.

### DESCRIPTION OF THE INVENTION

A particular object of the present invention is to provide a system and a method that allows detecting and transmitting information of a person's electrocardiogram, at any time and in any condition. Such objects can be reached by the present invention thanks to a system according to claim 1 and to a method according to claim 8. Further characteristics and advantages of the present invention are detailed in the respective claims.

In a first aspect of the present invention, a system for the detection and the processing of physical parameters of human body is provided, which includes a plurality of sensors applicable to the skin of the subject, characterized by the fact that it comprises at least one interface circuit for processing sensor outputs and at least one reception/transmission circuit for the transmission of the signals processed by the interface circuit.

In the preferred embodiment, signals detected by the sensors are signals of an electrocardiogram of the subject. The detection of other physical parameters, for example body temperature, arterial blood pressure and other, can be possibly provided, as long as specific sensors for such purpose are available.

In a particularly advantageous aspect of the present invention, the reception/transmission circuit is included in a cellular phone. Since a cellular phone is an everyday object, such a solution allows everyone to be able to notify conveniently and in a particularly simple way to a remote unit the arising of cardiac problems.

In a possible embodiment of said system, also the interface circuit is included in a cellular phone. Said cellular phone being therefore arranged to process signals received by the sensors and to transmit on request significant information to a particular remote equipment, which can be either a cellular phone or a landline telephony equipment.

In the preferred embodiment, the cellular phone includes at least a compartment in which sensors can be kept when they are not applied to the skin of the subject. In such a way, sensors necessary for signal detection can be always provided to the person that uses the cellular phone and do not encumber when they are not used. Alternatively, a compartment to keep sensors can be realized in the cellular phone case, provided that the subject uses one. In this way it is possible to avoid that the sensors being separated from the cellular phone and unintentionally placed where they do not come handy.

In a possible embodiment, the connection between the sensors and the cellular phone is wireless, for example infrared transmission or radio-frequency transmission. Alternatively, said connection can also be a wired connection, by providing the set of sensors with a single connector, which is compatible with a suitable data link socket of the cellular phone it is associated with.

In a second aspect of the present invention, a method for the detection and the processing of physical parameters of the human body is provided, in which a plurality of sensors is applied to the skin of a subject, characterized by the fact that it comprises the following phases:

a) detecting sensors signals;

b) processing the detected signals in order to obtain one or more images representative of the signals;

c) transmitting images to a remote unit as radio-frequency signals.

In particular, the signals detected are preferably at least signals of an electrocardiogram of the subject, such signals being transformed into images. An image advantageously provides an information which is particularly significant and immediately decipherable by the person who receives them. Images are preferably transmitted by a cellular phone to a remote unit, for example the cellular phone of a trusted physician or to a telephony equipment, for example a telephone or a mobile phone equipped with a visualization screen, a facsimile or similar, which is present in a diagnostic centre or in a centre equipped for telemedicine services. The processing of the images is preferably realized by a suitable interface circuit included in a cellular phone. Alternatively, said interface circuit can be included in an external unit which can be connected, with a wired or wireless connection, to the mobile phone and to the sensors.

In a third aspect of the present invention, a set of sensors for the detection and the processing of physical parameters of human body is provided, characterized by the fact that it comprises a plurality of sensors applicable to the skin of a subject, said plurality of sensors being assembled on a flexible or foldable support. Such support is preferably shaped, in order to facilitate the right application of the sensors to the skin of the subject and the electrical conductors of the sensors are grouped in a single connection at their free end, thus allowing the connection to an interface circuit or to a cellular phone.

In a possible embodiment, the set of sensors comprises at least six sensors for the detection of the signals of an electrocardiogram. Obviously, further sensors can be added, for example a seventh sensor still aimed at detecting signals of an electrocardiogram or other sensors aimed at detecting further significant parameters.

The “cardiac stripe” or “component with sequence of sensors on a flexible support” can be easily used by everyone, because it realizes in a single semi-adhesive element, which is stripe-shaped, the guided placement of several measure points, which are necessary for the detection of an electrocardiogram.

In fact, thanks to a proper shape of the said stripe, which can provide precise reference points on the user's body (for example, ribs, breast-bone, nipple, etc.), it is possible to apply said device in the correct way, without making any mistake. The flexible support comprises the proper spacing among each sensor, in such way allowing the guided positioning of the stripe. Said guided positioning and the adaptable conformations make said system quick and effective to apply, thus decreasing the sensor application time, as if the set of sensor was a single sensor. Furthermore, the connection system facilitates the system's substitution, thus allowing its use as a disposable item.

Said system can be applied also to standard instruments, thanks to connectors and/or interface circuits which connect, also wireless, the stripe to a standard electrocardiogram. Fitness characteristics are specified, which easily allow proper stretching and shortening of the flexible support, thanks to, as a non binding example, one or more foldings. Moreover, the stripe can comprise integrated, overturnable or connectable branches, which can reach other relevant points on the subject which are not in sequence.

### BRIEF DESCRIPTION OF THE DRAWINGS

Further advantages and characteristics of the present invention will become more readily appreciated by reference to the following description, made by way of example and not restrictive, when taken in conjunction with the accompanying drawings, wherein:

FIG. 1 is a schematic representation which generally illustrates the main idea of the invention;

FIG. 2 is a representation of a possible embodiment of the present invention;

FIG. 3 is a representation of a further possible embodiment of the present invention;

FIG. 4 is a schematic perspective view of a possible embodiment of a mobile phone suitable to the use in a system according to the present invention; and

FIG. 5 schematically illustrates a possible embodiment of a set of sensors according to the present invention.

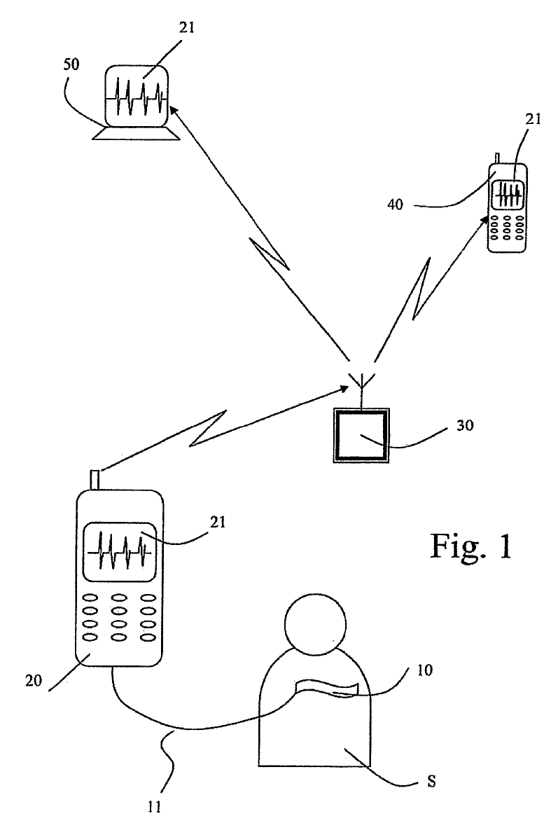

FIG. 1 shows a subject S, who has been applied a set of sensors 10 for the detection of signals of an electrocardiogram. The sensors of the set of sensors 10 are electrically connected to a cellular phone 20 by means of conductors 11 preferably joined in a single cable or stripe provided at its free end with a connector 12 (FIG. 5) suitable to the connection to a cellular phone 20. In the possible embodiment shown in FIG. 1, the cellular phone 20 includes an interface circuit which can process the signals coming from the set of sensors 10 in order to obtain an image 21 for each of the sensors V1-V6 (FIG. 5) which are present in the set of sensors 10. Each image 21 is stored, possibly visualized on the screen of the cellular phone 20, and transmitted over a cellular phone network for example a GSM network or similar here schematized in a simple manner by a single node 30, to a remote unit, like a cellular phone 40 belonging for example to a doctor or, alternatively or in combination, to the monitoring station 50 of a specialist centre provided with suitable telecommunications equipment or with systems for telemedicine.

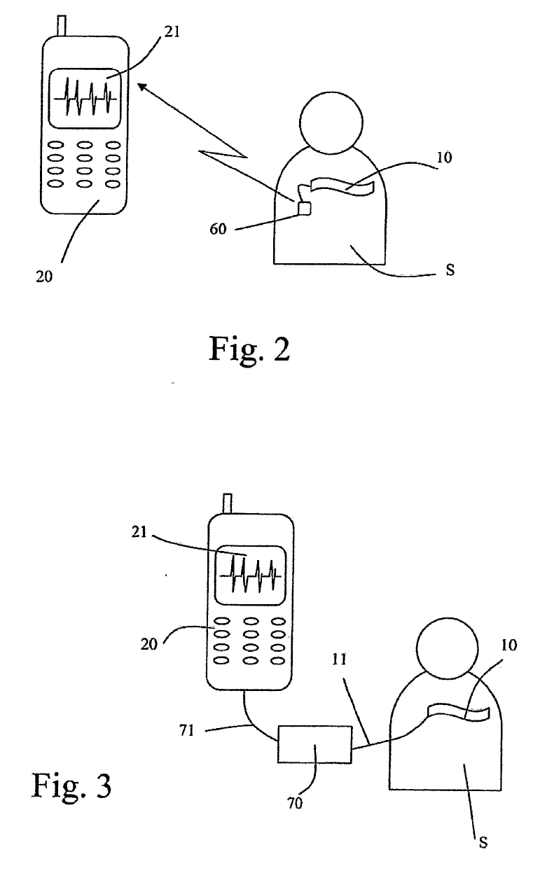

As shown in FIG. 2, the connection between the set of sensors 10 and the cellular phone 20 can be realized also wireless, for example by means of a suitable interface circuit 60 which uses for example radio frequency signals, infrared signals or similar (for example a BlueTooth® system or a wi-fi hot spot), in order to transmit the detected signals to the cellular phone 20.

FIG. 3 shows a possible embodiment where an interface circuit 70, which connects the set of sensors 10 to the cellular phone 20 is interposed. Said interface circuit 70 can directly process the received signals in order to produce images, possibly storing said images, and then transmit said images to the cellular phone 20. As an alternative, interface circuit 70 can also be arranged to perform only some of the signal processing operations (e.g., analog-to-digital conversion, storage and/or control of the data flow from the sensors to the cellular phone 20). Connection wires 11 and 71, which connect said interface circuit respectively with the set of sensors 10 and with the cellular phone 20, can obviously be replaced by a circuit like the one labeled 60 in FIG. 2 for wireless transmission of the signal.

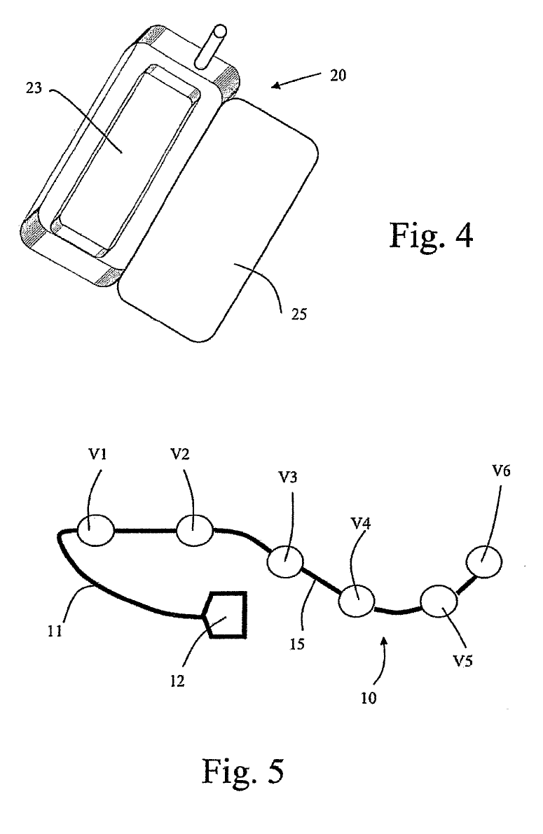

In an advantageous embodiment, schematically shown in FIG. 4, the cellular phone 20 can be provided, for example in the posterior part of its shell, with a compartment 23 into which at least the set of sensors 10 can be folded. If necessary, also the possible interface circuit 70 can be kept in the said compartment 23, if there is enough space. A cover 25 allows locking the access to the compartment 23 in order to keep the sensors and/or the interface circuit when they are not used.

In an alternative way or in combination, the sensors and the interface circuit can also be kept in a case (not represented) of the cellular phone 20.

In FIG. 5 a set of sensors 10, in which six sensors, labeled V1-V6, are provided, and assembled on a support 15 realized using a flexible and foldable material is shown. Said support 15 is preferably shaped in order to facilitate the right application of the sensors on the skin of the subject S, for example sensors VI and V2 being applied to the sides of the breast bone and sensors V3-V6 applied immediately under the pectoral muscle. Said support 15 encloses also the electrical conductors associated with each sensor, said electrical conductors being connected, at their free end, to a single connector 12, which allows the connection of the said set of sensors 10 to the cellular phone 20 and/or to an interface circuit like the said interface circuits labeled 60 (FIG. 2) and 70 (FIG. 3).

1-23. (canceled)

24: A system for the detection and processing of physical parameters of the human body, comprising:
a plurality of sensors applicable to the skin of a subject;
an interface circuit for the processing of signals coming from said sensors;
a reception/transmission circuit for the transmission of the signals processed by said interface circuit; and
a remote unit having a graphic display, said remote unit being adapted to receive said signals from said reception/transmission circuit and display said signals on said graphic display.

25: The system according to claim 24, wherein said signals detected by said sensors are signals of an electrocardiogram of the subject.

26: The system according to claim 24, wherein said reception/transmission circuit is included in a cellular phone.

27: The system according to claim 24, wherein said interface circuit is included in a cellular phone.

28: The system according to claim 26, wherein said cellular phone comprises at least one compartment adapted to retain said sensors when they are not applied to the skin of the subject.

29: The system according to claim 26, wherein said interface circuit includes wireless connections which allow the transmission of signal flow from said sensors to said cellular phone.

30: The system according to claim 24, wherein said sensors are provided on a flexible support grouped in a single connection at their free end in order to allow the connection of said plurality of sensors to a single connector, said flexible support being properly shaped to be flexible and extensible.

31: The system according to claim 30, wherein said sensors are at least six sensors applied in suitable positions on said flexible support so that at least two sensors are positionable to the sides of the breast bone of the subject.

32: A physical parameter detection and processing system comprising:
a cardiac stripe having at least one connector, and a sequence of sensors provided to a flexible support, said sensors are conductive and semi-conductive electrical elements in contact with the skin that can detect signals related to heart activity, said sensors are connected by means of conductive parts which are integrated in said flexible support, said flexible support comprises joints allowing said flexible support to reach suitable positions on the subject;
an interface circuit for the processing of signals coming from said sensors of said cardiac stripe;
at least a reception/transmission circuit included in a cellular phone, said cellular phone being adapted to receive said signals from said interface circuit and transmit them as radio-frequency signals; and
a remote unit having a reception system and a graphic display, said remote unit being adapted to receive said radio-frequency signals from said cellular phone and display said signals on said graphic display as an image.

33: The system according to claim 32, wherein said sensors are at least six sensors applied in suitable positions on said flexible support so that at least two sensors are positionable to the sides of the breast bone of the subject.

34: The system according to claim 32, wherein said cellular phone comprises at least one compartment and a compartment cover, said compartment being adapted to retain said sensors when they are not applied to the skin of the subject.

35: A method for the detection and processing of physical parameters of the human body, said method comprising the steps of:
providing a detection and processing system comprising: a plurality of sensors applicable to the skin of a subject; an interface circuit for the processing of signals coming from said sensors; a reception/transmission circuit for the transmission of the signals processed by said interface circuit; and a remote unit having a graphic display, said remote unit being adapted to receive said signals from said reception/transmission circuit and display said signals on said graphic display;
applying said sensor to the skin of a subject;
connecting said sensors to said interface circuit;
detecting output signals from said sensors;
processing said detected signals by means of said interface circuit to obtain at least one image representative of said signals;
transmitting said image to said reception/transmission circuit;
transmitting said image to said remote unit as radio-frequency signals; and
processing said radio-frequency signals so as to be displayed on said graphic display in said remote unit.

36: The method according to claim 35, wherein said signals detected by said sensors are signals of an electrocardiogram of the subject.

37: The method according to claim 35, wherein said image is an image for each of the said sensors is created.

38: The method according to claim 35, wherein said at least one image is transmitted by means of a cellular phone.

39: The method according to claim 38, wherein said processing of said image is realized by said interface circuit included in said cellular phone.

40: The method according to claim 38, wherein said sensors are storable in a compartment defined in said cellular phone when said sensors are not applied to the skin of the subject.

41: The method according to claim 35, where said remote unit is a cellular phone.

42: The method according to claim 35, where said remote unit is a centre for telemedicine services.
